(19) United States
(12) Patent Application Publication
(10) Pub. No.: US 2019/0346379 A1
(43) Pub. Date: Nov. 14, 2019

(54) X-RAY SCREENING SYSTEM AND METHOD

(71) Applicant: VOTI INC., St-Laurent (CA)

(72) Inventors: William AWAD, St-Laurent (CA); Emmanuel ST-AUBIN, St-Laurent (CA); Philippe DESJEANS-GAUTHIER, St-Laurent (CA); Simon ARCHAMBAULT, St-Laurent (CA); Nicolas TEYSSEDOU, St-Laurent (CA)

(21) Appl. No.: 16/406,295

(22) Filed: May 8, 2019

Related U.S. Application Data
(60) Provisional application No. 62/669,607, filed on May 10, 2018.

(51) Int. Cl. G01N 23/02; G01N 33/10

(52) U.S. Cl. CPC G01N 23/02; G01N 2223/1016; G01N 33/10

(57) ABSTRACT

An x-ray screening system includes a plurality of x-ray screening devices each for scanning at least one object of interest. Each screening device emits x-rays which pass through the object of interest and which are detected by a group of detectors including at least one detector to provide measured x-ray energy signals. At least one central processor is in data communication with each screening device for receiving the measured x-ray energy signals from each screening device automatically and in real-time. The at least one processor automatically analyzes the measured x-ray energy signals in real-time to determine at least one property of the object of interest and determining whether the at least one property indicates that at least a portion of the object of interest is composed of a material of interest. The material of interest may be a potentially dangerous material.

### CROSS-REFERENCE TO RELATED APPLICATIONS

This patent application is a non-provisional of and claims priority to and the benefit of U.S. Provisional Patent Application No. 62/669,607, filed on May 10, 2018, entitled “X-RAY SCREENING SYSTEM AND METHOD”, which is incorporated by reference in its entirety.

### FIELD OF THE INVENTION

The present invention relates to a system for screening of objects and materials using x-ray screening devices. More particularly, the present invention relates to a system for screening of objects and materials using a plurality of x-ray screening devices in data communication with a central processor.

### BACKGROUND

As a matter of public safety, some locations may be provided with screening devices such as x-ray screening devices for the purpose of scanning people and objects so as to identify and prevent the unauthorized passage of weapons, dangerous materials, contraband or other undesirable items into or out of the location. Such locations may include, for example, airports, entertainment events, shopping centers, prisons and other locations that may be accessible to large numbers of people. At these locations, the screening devices may be located at defined screening checkpoints. Moreover, security screening of materials and objects is often used at locations with high throughput, such as airports, where people and baggage must be scanned at a relatively high rate so as to avoid congestion at security checkpoints. At those checkpoints people and items are scanned. The scans are reviewed by security personnel and the person or item may be subject to further physical search.

In some systems, a person or object is scanned and an image is generated for review by human operator such as security personnel at an airport. In other systems, software may be used for the purpose of processing a generated or refined image to identify potentially threatening objects or materials. The software determines whether the pixels of the image represent an object or material of interest and the image may then be forwarded to a human operator for second-level screening. In both of these cases, there is introduced a “human intervention” step and therefore a step whereby human error may be introduced. For example, a human operator reviewing the image may fail to identify potentially threatening materials or objects contained in the image. There is also introduced a problem of limited throughput at screening stations due to the time required for security personnel to review the image flagged by the software, as well as the decision-making process for possible rerouting of personnel, baggage and/or passengers. Such human intervention may cause undue delay at the security screening checkpoint. There is an added issue wherein while security personnel review the information provided in the refined image, their attention is diverted away from their surroundings and therefore away from potentially threatening situations in their surroundings.

Some systems have been put in place to manage passenger throughput at airport security screening checkpoints. However, the reliance on refined image data and processing by human operators does not efficiently address the complications associated with steady throughput at security screening checkpoints.

Some systems have been introduced which include centralized review of refined images produced by scanning. However, due to a lack of integration between the scanning devices themselves, these systems fail to identify benign or non-threatening materials which could pass through a single checkpoint together in separate containers or through different checkpoints to later be combined to generate a dangerous material. This is of particular concern in the detection of liquid explosives.

In view the above, advantage would be found with an x-ray scanning system and method which provides for automatic, real-time, centralized analysis of data obtained by x-ray scanning devices at one or more screening checkpoints.

### BRIEF DESCRIPTION OF THE DRAWINGS

FIG. 1 is an illustration of an exemplary x-ray scanning device which may be used in association with the present invention;

FIG. 2 is an illustration of a system which may be used in association with the present invention; and,

FIG. 3 is an illustration of an exemplary cloud-based system for processing of information from a plurality of x-ray scanning devices.

### SUMMARY

The present invention relates to a system for screening of objects and materials using x-ray screening devices. More particularly, the present invention relates to a system for screening of objects and materials using a plurality of x-ray screening devices in data communication with a central processor.

In one aspect, there is provided an x-ray screening system including a plurality of x-ray screening devices each for scanning at least one object of interest. Each x-ray screening device emits x-rays which pass through the object of interest and which are detected by a group of detectors including at least one detector to provide measured x-ray energy signals. At least one central processor is in data communication with each x-ray screening device of the plurality of x-ray screening devices for receiving the measured x-ray energy signals from each x-ray screening device of the plurality of x-ray screening devices automatically and in real-time. The at least one central processor automatically analyzes the measured x-ray energy signals in real-time to determine at least one property of the object of interest scanned by at least one of the plurality of x-ray screening devices and determines whether the at least one property indicates that at least a portion of the object of interest is composed of a material of interest. The at least one property may be at least one of a physical property and a chemical property. The at least one property may include at least one of atomic number, effective atomic number, mass thickness, density and mass density.

The material of interest may be a potentially dangerous material. The material of interest may also include at least one material which may be combined with at least one other material to create at least one third material that is potentially dangerous.

An alert module may be coupled with at least one of the central processor and at least one x-ray screening device of the plurality of x-ray screening devices for raising an alert condition when the at least a portion of the object of interest is composed of the material of interest.

The system may include means for comparing the at least one property with a predetermined safe metric and a predetermined threat metric, and, where the at least one property surpasses a predetermined threshold for the threat metric, raising the alert condition and where the at least one property is below the predetermined threshold, not raising the alert condition.

At least one local processor may be coupled with an x-ray screening device of the plurality of the x-ray screening devices for processing the measured x-ray signals locally at the x-ray screening device.

The system may include a background removal module for processing the x-ray signals to identify x-ray signals which do not represent the object of interest and remove from the x-ray signals the x-ray signals which do not represent the object of interest.

The system may include an object reconstruction module for processing the x-ray signals to reconstruct an image of the object of interest absent x-ray signals which do not represent the object of interest.

The system may include a reference material decomposition module for processing the measured x-ray energy signals of the object of interest into known x-ray energy information of one or more known materials.

Each of the x-ray screening devices and the at least one central processor are each components of a cloud-based system.

The at least one central processor may be in data communication with at least one x-ray screening device of the plurality of x-ray screening devices for transmitting at least one of analyzed measured x-ray signal data and the alert condition to the at least one x-ray screening device.

In another aspect, there is provided an x-ray screening method including the steps of providing a plurality of x-ray screening devices, scanning at least one object of interest in at least one x-ray screening device of the plurality of x-ray screening devices, measuring x-ray energy signals provided by emitted x-rays which pass through the object of interest and which are detected by a group of detectors including at least one detector, communicating the measured x-ray signals from each x-ray screening device of the plurality of x-ray screening devices automatically and in real-time to at least one central processor in data communication with each x-ray screening device of the plurality of x-ray screening devices, automatically analyzing the measured x-ray energy signals in real-time, determining at least one property of the object of interest scanned by at least one of the plurality of x-ray screening devices, and, determining whether the at least one property indicates that at least a portion of the object of interest is composed of a material of interest. The at least one property may be at least one of a physical property and a chemical property. The at least one property may include at least one of atomic number, effective atomic number, mass thickness, density and mass density.

The material of interest may be a potentially dangerous material. The material of interest may also include at least one material which may be combined with at least one other material to create at least one third material that is potentially dangerous.

An alert condition may be raised where the at least a portion of the object of interest is composed of the material of interest.

The measured x-ray signals may be processed locally at the x-ray screening device using at least one local processor coupled with an x-ray screening device of the plurality of the x-ray screening devices.

The method may further include the steps of processing the x-ray energy signals to identify x-ray energy signals which do not represent the object of interest, and, removing from the x-ray energy signals the identified x-ray energy signals which do not represent the object of interest.

The method may further include the steps of processing the x-ray signals to construct an image of the object of interest absent x-ray signals which do not represent the object of interest.

The method may further include the steps of comparing the at least one property with a predetermined safe metric and a predetermined threat metric, and, where the at least one property surpasses the predetermined threshold for the threat metric, raising the alert condition and where the at least one property is below the predetermined threshold, not raising the alert condition.

The method may further include the steps of decomposing the measured x-ray energy signals of the object of interest into known x-ray energy information of one or more known materials.

Each of the x-ray screening devices and the at least one central processor are each components of a cloud-based system.

There is provide an x-ray scanning system and method which provides for automatic, real-time, centralized analysis of data obtained by x-ray scanning devices at one or more screening checkpoints.

### DESCRIPTION

The present invention relates to a system for screening of objects and materials using x-ray screening devices. More particularly, the present invention relates to a system for screening of objects and materials using a plurality of x-ray screening devices in data communication with a central processor.

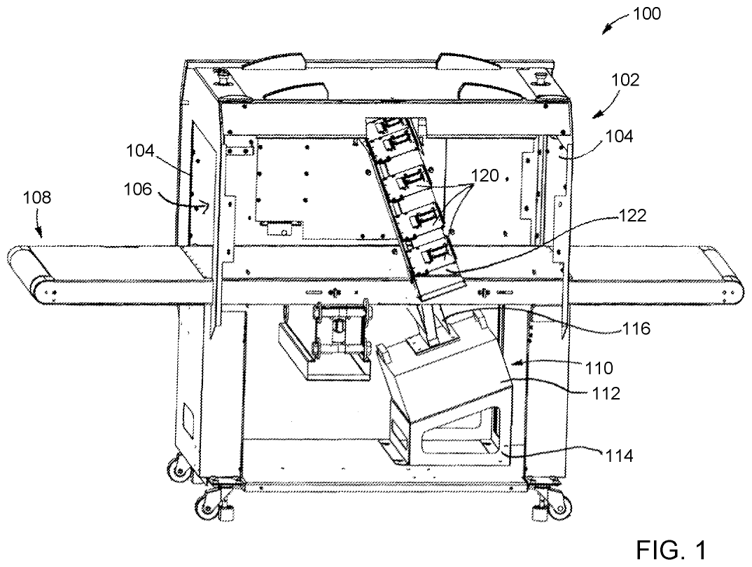

According to the aspect shown in FIG. 1, there is provided an exemplary x-ray scanning device 100. The x-ray scanning device 100 includes a housing 102 having openings 104 at either end thereof. The openings 104 provide access to a scanning chamber 106 passing through the housing 102. The system 100 may further include a displacement assembly 108, such as a conveyor, which extends through the scanning chamber 106 and which may be used to displace at least one object of interest to be scanned using the x-ray scanning device 100. The x-ray scanning device 100 further includes a source assembly 110. The source assembly 110 includes a source (not shown) for emitting electromagnetic radiation such as x-rays, a source assembly housing 112 at least partially enclosing the source, a pedestal 114 to which the source assembly housing 112 is mounted and a collimator 116 mounted to the source assembly housing 112 for directing x-rays emitted from the source. Collimator 116 may for example be a fan-shaped collimator for directing the x-rays in a fan-shaped beam. It should be understood that collimator 116 may be of any suitable shape and not only fan-shaped.

The x-ray scanning device 100 may further include a group of detectors including at least one detector 120 and preferably a plurality of detectors 120 each mounted to the bracket 122. In one aspect, the bracket is an L-shaped bracket which is positioned within the scanning chamber 106 such that the plurality of detectors 120 are mounted at least partially about the scanning chamber 106. In the aspect shown in FIG. 1 there is shown mounted within the scanning chamber a single bracket 122. It should be understood that in other aspects, the scanning chamber may include more than one bracket positioned within the scanning chamber and that the brackets do not have to have same orientation or angular position. It should be further understood that the bracket 122 does not have to be L-shaped. Rather, the bracket 122 may be linear or arc shaped or any other suitable shape.

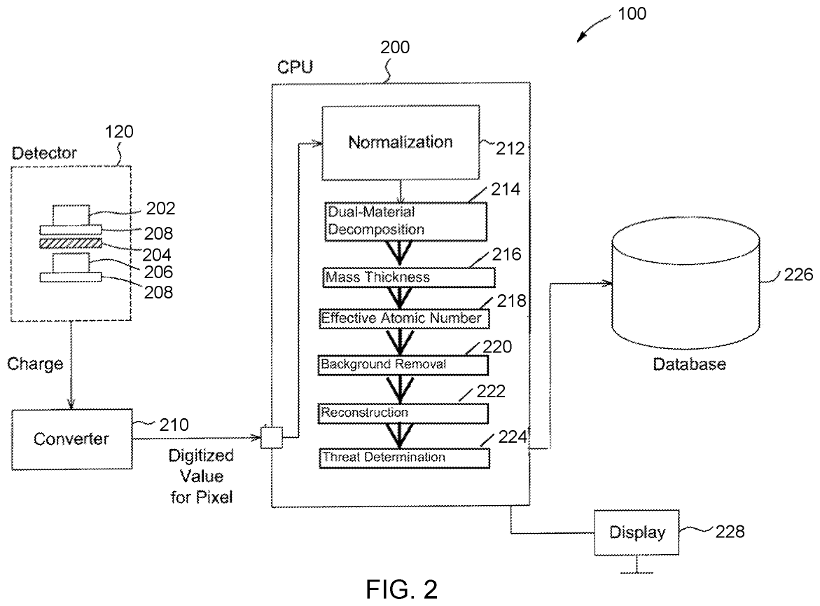

In some embodiments, each detector 120 includes a detector card having a center point and edges. The center point corresponds to the geometric center of the detector cards. The edges of each detector card define the boundaries of the detector 120. As shown in FIG. 2, detectors 120 and the x-ray scanning device 100 may be linked to a local central processing unit (CPU) 200 or other local processing device coupled with the x-ray scanning device so that x-ray signals detected by the detectors 120 may be analyzed locally, processed locally, and used to output information locally. Such output may include output of an image to a display for review by security personnel.

In the context of the present description, the term “processor” refers to at least one computerized component for executing computer-executable instructions. This may include, for example, a central processing unit (CPU), a microprocessor, a controller, and/or the like. A plurality of such processors may be provided, according to different aspects of the present invention, as can be understood by a person skilled in the art. The processor may be provided within one or more general-purpose computers, for/or any other suitable computing device.

The term “storage” may refer to any computer data storage device or assembly of such devices including, for example, a temporary storage unit such as random-access memory (RAM) or dynamic RAM, permanent storage medium such as a hard disk, and optical storage device, such as a CD or DVD (rewritable or write once/read only), a flash memory, a database, and/or the like. A plurality of such storage devices may be provided, as can be understood by a person skilled in the art.

According to the aspect shown in FIG. 2, each detector 120 may comprise a first scintillator 202, a filter 204 and a second scintillator 206. All of these may be sandwiched together or otherwise suitably arranged as shown in FIG. 2. In a scanning operation, broad-spectrum x-rays are emitted by the source and are directed by the collimator 116 toward the plurality of detectors 120 within the scanning chamber 106. In the case of each detector 120, a plurality of the emitted x-rays encounter the first scintillator 202 which may be configured to detect the lower portion of the emitted x-ray signal spectrum. Residual low energy x-ray signals may then be stopped by the filter 204 and remaining x-ray signals from the emitted x-rays reach the second scintillator 206 which may be configured to detect a higher portion of the x-ray signal spectrum.

With further reference to FIG. 2, in one aspect, each of the scintillators 202, 206 converts the detected x-ray energy to light. Each of these scintillators 202, 206 is coupled with a photodiode 208 which captures the light from the respective scintillator 202, 206 and generates a corresponding analog electric signal. The electric signal is further digitized by a converter 210. The digitized signal value is associated with a pixel of an image for providing a visual representation of an object being scanned.

In the conversion of the light into an electric signal by the photodiodes 208, some uncertainties may be introduced, in that a given light source may result in different electric signals due to the fact that every detector card reacts slightly differently to the presence or absence of the electromagnetic radiation of an x-ray. In order to correct these variations and for the final image to appear more homogenously, each pixel of the image may be normalized by correcting an offset and gain in the light conversion. Such a normalization procedure may be executed for example using a normalization module 212 as shown in FIG. 2 in order to compensate for slight variations in offset and gain for each detector, as well as for estimating the expected uncertainties in the low-energy and high-energy signals and/or attenuation for each detector.

The apparatus may further include a reference-material or dual-material decomposition module 214 for decomposing x-ray energy information of an unknown object or material in terms of known x-ray energy information of one or more known objects or materials, a mass thickness determination module 216 for determining the mass thickness of one or more objects or materials of interest, an effective atomic number module 218 for determining the effective atomic number of one or more objects or materials of interest, a background removal module 220 for processing the high and low energy x-ray signals to identify x-ray signals which do not necessarily represent the object of interest and remove from the x-ray signals the identified x-ray signals which do not represent the object of interest, a reconstruction module 222 for processing the x-ray signals to reconstruct an image of the object of interest absent identified not-of-interest x-ray signals which do not represent the object of interest and a threat determination module 224 for determining whether one or more objects or materials of interest pose a threat and correspondingly raising an alarm or alert condition based on the determination. Information acquired by any of the aforementioned modules may be saved to a suitable storage medium such as a database226. Moreover, images may be output to a display 228.

A system of one or more computers can be configured to perform the particular operations or actions as described herein by virtue of having software, firmware, hardware, or a combination of them installed on the system that in operation causes or cause the system to perform the actions. One or more computer programs can be configured to perform particular operations or actions described herein by virtue of including instructions that, when executed by data processing apparatus, cause the apparatus to perform the actions.

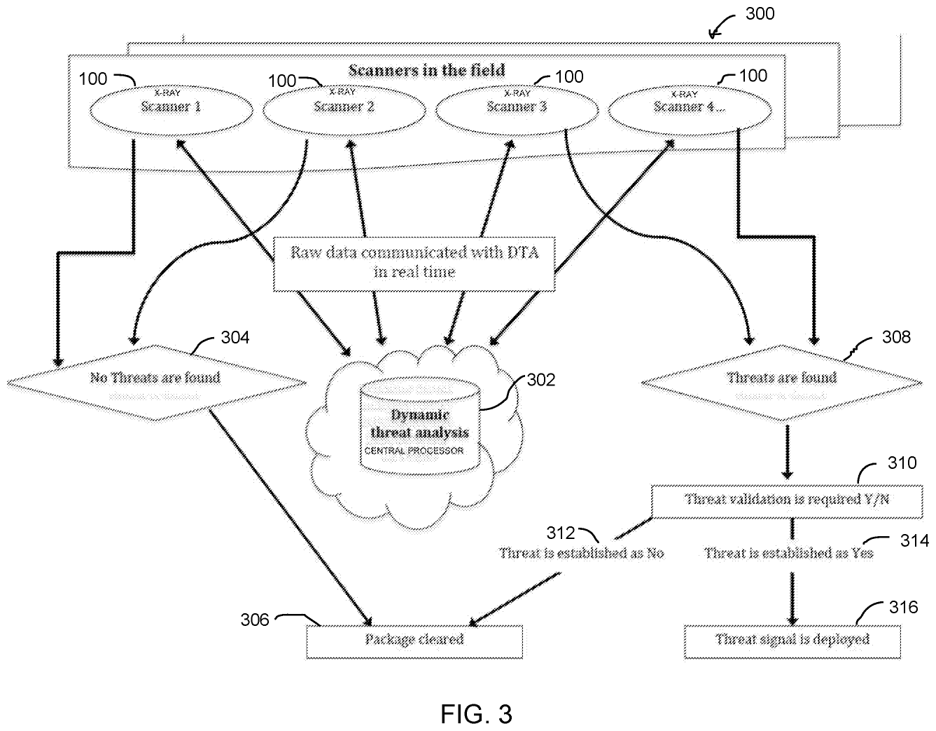

In one general aspect shown in FIG. 3, there is provided an x-ray screening system 300 including a plurality of x-ray screening devices 100 each for scanning at least one object of interest. Each x-ray screening device 100 emits x-rays which pass through the object of interest and which are detected by a group of detectors including at least one detector to provide measured x-ray energy signals. The system also includes at least one central processor 302 in data communication with each x-ray screening device 100 of the plurality of x-ray screening devices 100 for receiving the measured x-ray energy signals from each x-ray screening device 100 of the plurality of x-ray screening devices 100 automatically and in real-time. The at least one central processor 302 analyzes the measured x-ray energy signals automatically and in real-time to determine at least one property of the object of interest scanned by at least one of the plurality of x-ray screening devices 100 and determining whether the at least one property indicates that at least a portion of the object of interest is composed of a material of interest. This information may be directed, in real-time, to one or more of the x-ray scanning devices 100. Where no threats are found, as shown at 304, the object of interest may be cleared as shown at 306. Alternatively, where the object of interest is found to include a material of interest as shown at 308, a threat validation may be undertaken as shown at 310. If the material of interest is not found to be a potential threat as shown at 312, then the object of interest may be cleared at 306. Where the material of interest is found to be a threat or a potential threat as shown at 314, then an alert condition may be raised as shown at 316.

Other embodiments of this aspect include corresponding computer systems, apparatus, and computer programs recorded on one or more computer storage devices, each configured to perform the actions of the methods. It should be further understood that the central processor and its associated architecture may be used to implement any of the processes or methods described herein as well as one or more of their associated steps either automatically or in real-time or both.

In one aspect, the x-ray screening devices and the at least one central processor may be in data communication with each other by way of a network interface which may establish and/or provide network connectivity to a network (e.g., a local area network, a wide area network, such as the Internet, etc.). The network interface may include hardware and/or software components for communicating via Ethernet, TCP/IP, FTP, HTTP, HTTPS, and/or other protocols. Similarly, a wireless interface may be relied upon to establish and/or provide network connectivity to a wireless network (e.g., a local area network, a wide area network, such as the Internet, a cellular voice and/or data network, etc.). Wireless interface 112 thus may include hardware and/or software components for communicating via Ethernet, TCP/IP, FTP, HTTP, HTTPS, IEEE 802.11b/g/a/n/ac, Bluetooth, CDMA, TDMA, GSM and/or other protocols. In a preferred embodiment, the x-ray screening devices and the at least one processor are at least a portion of a “cloud” based system wherein processing of information from each of the x-ray scanning devices may be centrally processed and analyzed either located with the x-ray scanning devices or remotely from the x-ray scanning devices and automatically and in real time.

The x-ray scanning system may further include an alert module coupled with the central processor for raising an alert condition where the at least one property indicates that at least a portion of the object of interest is composed of a material of interest. Such an alert condition may be raised, for example, where the at least one property is compared with a predetermined safe metric and a predetermined threat metric. Where the at least one property surpasses a predetermined threshold for the threat metric the alert condition may be raised and where the at least one property is below the predetermined threshold, the alert condition may not be raised.

Materials of interest may fall into a known or predetermined risk family of materials, which may include at least one potentially dangerous material or at least one material which may be combined with at least one other material to create at least one third material that is potentially dangerous. The at least one property may be at least one of a chemical property and a physical property. The at least one property may include at least one of atomic number, effective atomic number, mass thickness, density and mass density.

In another aspect, as previously described, the x-ray screening system may include at least one local processor coupled with an x-ray screening device of the plurality of the x-ray screening devices for processing the measured x-ray energy signals locally at the x-ray screening device.

In another aspect, the x-ray scanning system may include a background removal module 220 and an object reconstruction module 222.

In another aspect, at least one of the x-ray screening devices of the plurality of x-ray screening devices may be a calibrated x-ray screening device for determining first and second basis material path lengths from the measured x-ray energy signals.

In another aspect, the at least one central processor is in data communication with at least one x-ray screening device of the plurality of x-ray screening devices to automatically transmit at least one of analyzed measured x-ray signal data and the alert condition to the at least one x-ray screening device.

One general aspect includes an x-ray screening method including the steps of providing a plurality of x-ray screening devices. The x-ray screening method also includes scanning at least one object of interest in at least one x-ray screening device of the plurality of x-ray screening devices, measuring x-ray energy signals provided by emitted x-rays which pass through the object of interest and which are detected by a group of detectors including at least one detector, communicating the measured x-ray energy signals from each x-ray screening device of the plurality of x-ray screening devices automatically and in real-time to at least one central processor in data communication with each x-ray scanning device of the plurality of x-ray screening devices, automatically analyzing the measured x-ray energy signals in real-time, determining at least one property of the object of interest scanned by at least one of the plurality of x-ray screening devices, and determining whether the at least one property indicates that at least a portion of the object of interest is composed of a material of interest. This information may be directed, in real-time, to one or more of the x-ray screening devices. Where no threats are found, the object of interest may be cleared. Alternatively, where the object of interest is found to include a material of interest, a threat validation may be undertaken. If the material of interest is not found to be a potential threat, then the object of interest may be cleared. Where the material of interest is found to be a threat or a potential threat, then an alert condition may be raised.

While the invention has been described in terms of specific embodiments, it is apparent that other forms could be adopted by one skilled in the art. For example, the methods described herein could be performed in a manner which differs from the embodiments described herein. The steps of each method could be performed using similar steps or steps producing the same result but which are not necessarily equivalent to the steps described herein. Similarly, the systems described herein could differ in appearance and construction from the embodiments described herein, the functions of each component of the systems could be performed by components of different construction but capable of a similar though not necessarily equivalent function, and appropriate materials could be substituted for those noted. Accordingly, it should be understood that the invention is not limited to the specific embodiments described herein. It should also be understood that the phraseology and terminology employed above are for the purpose of disclosing the illustrated embodiments, and do not necessarily serve as limitations to the scope of the invention. Finally, while the appended claims recite certain aspects believed to be associated with the invention, they do not necessarily serve as limitations to the scope of the invention.

1. An x-ray screening system comprising:
a plurality of x-ray screening devices each for scanning at least one object of interest, each x-ray screening device emitting x-rays which pass through the object of interest and which are detected by a group of detectors including at least one detector to provide measured x-ray energy signals;
at least one central processor in data communication with each x-ray screening device of the plurality of x-ray screening devices for receiving the measured x-ray energy signals from each x-ray screening device of the plurality of x-ray screening devices automatically and in real-time; wherein,
the at least one central processor automatically analyzes the measured x-ray energy signals in real-time to determine at least one property of the object of interest scanned by at least one of the plurality of x-ray screening devices and determining whether the at least one property indicates that at least a portion of the object of interest is composed of a material of interest.

2. The x-ray screening system according to claim 1, wherein the material of interest is a potentially dangerous material.

3. The x-ray screening system according to claim 1, further comprising:
an alert module coupled with at least one of the central processor and at least one x-ray screening device of the plurality of x-ray screening devices for raising an alert condition when the at least a portion of the object of interest is composed of the material of interest.

4. The x-ray screening system according to claim 1, wherein the material of interest includes at least one material which may be combined with at least one other material to create at least one third material that is potentially dangerous.

5. The x-ray screening system according to claim 1, further comprising:
at least one local processor coupled with an x-ray screening device of the plurality of the x-ray screening devices for processing the measured x-ray signals locally at the x-ray screening device.

6. The x-ray screening system according to claim 1, wherein the at least one property includes at least one of atomic number, effective atomic number, mass thickness, density and mass density.

7. The x-ray screening system according to claim 1, further comprising:
a background removal module for processing the x-ray signals to identify x-ray signals which do not represent the object of interest and remove from the x-ray signals the x-ray signals which do not represent the object of interest.

8. The x-ray screening system according to claim 6, further comprising:
an object reconstruction module for processing the x-ray signals to reconstruct an image of the object of interest absent x-ray signals which do not represent the object of interest.

9. The x-ray screening system according to claim 3, further comprising:
means for comparing the at least one property with a predetermined safe metric and a predetermined threat metric; and, where the at least one property surpasses a predetermined threshold for the threat metric, raising the alert condition and where the at least one property is below the predetermined threshold, not raising the alert condition.

10. The x-ray screening system according to claim 1, further comprising:
a reference material decomposition module for processing the measured x-ray energy signals of the object of interest into known x-ray energy information of one or more known materials.

11. The x-ray screening system according to claim 1, wherein the property is at least one of a chemical property and a physical property.

12. The x-ray screening system according to claim 1, wherein each of the x-ray screening devices and the at least one central processor are each components of a cloud-based system.

13. The x-ray screening system according to claim 3, wherein the at least one central processor is in data communication with at least one x-ray screening device of the plurality of x-ray screening devices for transmitting at least one of analyzed measured x-ray signal data and the alert condition to the at least one x-ray screening device.

14. An x-ray screening method comprising the steps of:
providing a plurality of x-ray screening devices;
scanning at least one object of interest in at least one x-ray screening device of the plurality of x-ray screening devices, measuring x-ray energy signals provided by emitted x-rays which pass through the object of interest and which are detected by a group of detectors including at least one detector;
communicating the measured x-ray signals from each x-ray screening device of the plurality of x-ray screening devices automatically and in real-time to at least one central processor in data communication with each x-ray screening device of the plurality of x-ray screening devices;
automatically analyzing the measured x-ray energy signals in real-time;
determining at least one property of the object of interest scanned by at least one of the plurality of x-ray screening devices; and,
determining whether the at least one property indicates that at least a portion of the object of interest is composed of a material of interest.

15. The x-ray screening method according to claim 14, wherein the material of interest is a potentially dangerous material.

16. The x-ray screening method according to claim 14, further comprising the step of:
raising an alert condition where the at least a portion of the object of interest is composed of the material of interest.

17. The x-ray screening method according to claim 14, wherein the material of interest includes at least one material which may be combined with at least one other material to create at least one third material that is potentially dangerous.

18. The x-ray screening method according to claim 14, further comprising the steps of:
processing the measured x-ray signals locally at the x-ray screening device using at least one local processor coupled with an x-ray screening device of the plurality of the x-ray screening devices.

19. The x-ray screening method according to claim 14, wherein the at least one property includes at least one of atomic number, effective atomic number, mass thickness, density and mass density.

20. The x-ray screening method according to claim 14, further comprising the steps of:
processing the x-ray energy signals to identify x-ray energy signals which do not represent the object of interest; and,
removing from the x-ray energy signals the identified x-ray energy signals which do not represent the object of interest.

21. The x-ray screening method according to claim 19, further comprising the steps of:
processing the x-ray signals to construct an image of the object of interest absent x-ray signals which do not represent the object of interest.

22. The x-ray screening method according to claim 16, further comprising the steps of:
comparing the at least one property with a predetermined safe metric and a predetermined threat metric; and,
where the at least one property surpasses the predetermined threshold for the threat metric, raising the alert condition and where the at least one property is below the predetermined threshold, not raising the alert condition.

23. The x-ray screening method according to claim 14 further comprising the step of:
decomposing the measured x-ray energy signals of the object of interest into known x-ray energy information of one or more known materials.

24. The x-ray screening method according to claim 14 wherein the property is at least one of a chemical property and a physical property.

25. The x-ray screening method according to claim 14 wherein each of the x-ray screening devices and the at least one central processor are each components of a cloud-based system.
